(19) United States
(12) Patent Application Publication
(10) Pub. No.: US 2003/0000027 A1
(43) Pub. Date: Jan. 2, 2003

(54) Novel colorant combination

(76) Inventors: Horst Hoeffkes, Duesseldorf (DE); David Rose, Hilden (DE); Bernd Meinigke, Leverkusen (DE)

Correspondence Address: HENKEL CORPORATION, 2500 RENAISSANCE BLVD, STE 200, GULPH MILLS, PA, 19406, US

(21) Appl. No.: 10/177,002

(22) Filed: Jun. 21, 2002

Related U.S. Application Data
(63) Continuation of application No. PCT/EP00/12819, filed on Dec. 15, 2000.

(30) Foreign Application Priority Data
Dec. 24, 1999 (DE) 199 62 871.8

(51) Int. Cl. A61K 7/13

(52) U.S. Cl. 8/405; 8/406; 8/407; 8/408; 8/415; 8/421

(57) ABSTRACT

New oxidation colorant compositions are provided containing a combination of at least one 4,5-diaminopyrazole derivative as a primary intermediate and at least one halogenated m-aminophenol as a secondary intermediate.

### CROSS REFERENCE TO RELATED APPLICATIONS

This application is a continuation under 35 U.S.C. §365(c) and §120 of International Application No. PCT/EP00/12819 filed Dec. 15, 2000 and under §119 of German Patent Application No. 199 62 871.8 filed Dec. 24, 1999.

### SUMMARY OF THE INVENTION

This invention relates to a new oxidation colorant for coloring keratin-containing fibers which contains a combination of at least one 4,5-diaminopyrazole derivative as primary intermediate and at least one halogenated m-aminopenol as secondary intermediate and to a process for coloring keratin fibers.

Nowadays, human hair is treated in many different ways with hair-care preparations. Such treatments include, for example, the cleaning of hair with shampoos, the care and regeneration of hair with rinses and conditioners and the bleaching, coloring and shaping of hair with coloring and tinting formulations, wave formulations and styling preparations. Among these, formulations for modifying or shading the color of the hair occupy a prominent position. Conventional hair colorants are formulated either on the basis of oxidation dyes or on the basis of substantive dyes according to the required color or the permanence thereof. In many cases, combinations of oxidation dyes and substantive dyes are also used to obtain special shades.

Colorants or tints containing substantive dyes as their coloring component are normally used for temporary colors. Substantive dyes are based on dye molecules which are directly absorbed onto the hair and do not require an oxidative process for developing the color. Dyes such as these include, for example, henna which has been used since ancient times for coloring the body and hair. Corresponding colors are generally far more sensitive to shampooing than the oxidative colors so that an often unwanted change of shade or even a visible “decoloration” then occurs very much more quickly.

So-called oxidation colorants are used for permanent, intensive colors with corresponding fastness properties. Oxidation colorants normally contain oxidation dye precursors, so-called primary intermediates and secondary intermediates. The primary intermediates form the actual dyes with one another or by coupling with one or more secondary intermediates under the influence of oxidizing agents or atmospheric oxygen. Oxidation colorants are distinguished by excellent long-lasting coloring results.

Good oxidation dye precursors are expected to satisfy above all the following requirements: they must form the required color tones with sufficient intensity and fastness during the oxidative coupling reaction. In addition, they must be readily absorbed onto the fibers with no significant differences—particularly in the case of human hair—between damaged and freshly regrown hair (levelling behavior). They are expected to be fast to light, heat and the effect of chemical reducing agents, for example permanent wave lotions. Finally, if they are used to color hair, they should not overly stain the scalp and, above all, should be toxicologically and dermatologically safe. It is also of advantage if the substances are readily soluble in various basic formulations. In addition, the color obtained, for example by blonding, should be easily removable from the hair if it does not meet the individual wishes of the user and is to be taken out.

In general, natural hair colors cannot be obtained with a primary intermediate alone or with a special secondary intermediate/primary intermediate combination. In practice, therefore, combinations of various primary intermediates and/or secondary intermediates are used. In addition, substantive dyes are often used for shading purposes. Accordingly, there is a constant demand for new improved dye combinations.

The problem addressed by the present invention, therefore, was to provide new combinations of dye precursors which would be suitable for use in colorants and which would meet the requirements they are expected to satisfy to a particular degree.

It has now surprisingly been found that combinations of special halogenated m-aminophenols as secondary intermediates with 4,5-diaminopyrazoles as primary intermediates meet the requirements oxidation colorants are expected to satisfy to a particularly high degree. Thus, brilliant, very intensive color tones in the red and magenta range which are extremely fast to light and washing are obtained using these secondary intermediates with, for example, 4,5-diamino-1-(2′-hydroxyethyl)-pyrazole as primary intermediate.

### BACKGROUND OF THE INVENTION

The use of 4,5-diaminopyrazole derivatives as primary intermediates in hair colorants is described, for example, in EP-B1-0 740 931. The use of other derivatives is known from WO 94108970 A1, WO 94/08969 A1 and DE 38 43 892 A1. The halogenated m-aminophenols used in the present invention are described as secondary intermediates in hair colorants in DE 196 07 158 A1 and DE 197 17 293 A1. However, these documents do not point to the excellent coloring properties of the combinations of 4,5-diaminopyrazoles with the special halogenated m-aminophenols according to the invention.

### DESCRIPTION OF THE INVENTION

In a first embodiment, therefore, the present invention relates to an oxidation colorant for coloring keratin-containing fibers which contains a combination of at least one 4,5-diaminopyrazole derivative or a physiologically compatible salt thereof as primary intermediate and at least one halogenated m-aminophenol corresponding to general formula (I) or a physiologically compatible salt thereof as secondary intermediate:

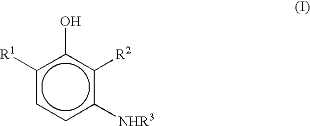

in formula (I):

R¹ is a C₁₋₄ alkyl group, a C₁₋₄ alkoxy group or an allyl group, R² is a halogen atom and R³ is hydrogen, a C₁₋₄ alkyl group, a C₁₋₄ alkoxy group, a C₃₋₄ dihydroxyalkyl group, a C₁₋₄ alkyl group substituted by 1 to 4 halogen atoms, a methanesulfonyl group, a group —COOR⁴, where R⁴ is hydrogen or a C₁₋₄ alkyl group, a group —COR_{f}, where R_{f} is a perfluorinated C₁₋₄ alkyl group, a group —CONR⁵R⁶ or a group —(CₙH₂ₙ)—B where n=1-4 and B is selected from a hydroxy group, a group —NR⁷R⁸ or a group —CONR⁹R¹⁰, R⁵, R⁶, R⁷, R⁸, R⁹ and R¹⁰ independently of one another representing hydrogen, a C₁₋₄ alkyl group or a C₁₋₄ monohydroxyalkyl group and R⁵ and R⁶, R⁷ and R⁸ and R⁹ and R¹⁰ together with the nitrogen atom carrying them may also be part of a morpholino, piperidino or pyrrolidino ring.

Examples of the C₁₋₄ alkyl groups mentioned as substituents in the compounds according to the invention are the methyl, ethyl, propyl, isopropyl and butyl groups. Ethyl and methyl are preferred alkyl groups, the methyl group being particularly preferred. According to the invention, a preferred C₁₋₄ alkoxy group is, for example, a methoxy group or an ethoxy group. Examples of a C₁₋₄ monohydroxyalkyl group are the hydroxymethyl or hydrozyethyl group. A 2-hydroxyethyl group is particularly preferred. According to the invention, examples of a halogen atom are a fluorine, chlorine, bromine or iodine atom, a chlorine atom being particularly preferred. An example of a C₁₋₄ alkyl group substituted by 1 to 4 halogen atoms, more particularly fluorine atoms, is a methyl group contaiing one to three halogen atoms, more particularly fluorine atoms, as substituents. According to the invention, halogenated C₁₋₄ alkyl groups terminated by a trifluromethyl group are particularly preferred.

The m-aminophenol derivatives corresponding to formula (I) where R² is chlorine have proved to be particularly suitable for the purposes of the invention.

Compounds of formula (I) in which R¹ is a C₁₋₄ alkyl group or an allyl group are also preferred for the purposes of the invention. Compounds containing a methyl group as R¹ have proved to be particularly suitable.

Other preferred compounds corresponding to general formula (I) are those in which R³ is hydrogen, a C₁₋₄ alkyl group, a trifluoromethyl group, a methanesulfonyl group, a group —COOR⁴, where R⁴ is hydrogen or a C₁₋₄ alkyl group, a group —COR_{f}, where R_{f} is a perfluorinated C₁₋₄ alkyl group, a group —CONR⁵R⁶ or a group —(CₙH₂ₙ)—B where n=1-4 and B is selected from a hydroxy group, a group —NR⁷R⁸ or a group —CONR⁹R¹⁰, R⁵, R⁶, R⁷, R⁸, R⁹ and R¹⁰ independently of one another representing hydrogen or a C₁₋₄ alkyl group.

According to the invention, a particularly preferred embodiment of the group —COR^{f} is the group —COCF₃. Particularly preferred embodiments of the group —(CₙH₂ₙ)—B are the groups —CH₂CH₂OH, —CH₂CH₂NH₂ and —CH₂CON(C₂H₅)₂. Other particularly preferred compunds are those in which R³ is the group —COOC₂H₅.

Secondary intermediates eminently suitable for the purposes of the invention as a whole are 2-chloro-6-methyl-3-(2′-hydroxyethylamino)-phenol, 2-chloro-6-methyl-3-(carbethoxyamino)-phenol, 4-(N′,N′-diethylcarbamoyl)-methylamino-2-hydroxy-3-chlorotoluene, 3-(2′-aminoethylamino)-2-chloro-6-methylphenol and 2-chloro-6-methyl-3-(trifluoroacetylamino)-phenol. 2-Chloro-6-methyl-3-(carbethoxyamino)-phenol, 2-chloro-6-methyl-3-(trifluoroacetylamino)-phenol, 4-(N′,N′-diethylcarbamoyl)-methylamino-2-hydroxy-3-chlorotoluene or a physiologically compatible salt of these compounds is most particularly preferred.

According to the invention, the primary intermediate preferably used is a 4,5-diaminoyrazole corresponding to general formula (II):

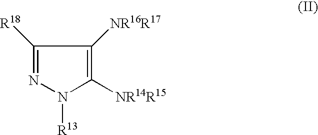

in formula (II):

R¹³, R¹⁴, R¹⁶ and R¹⁷ independently of one another represent hydrogen, a linear or branched C₁₋₆ alkyl group, a C₂₋₄ hydroxyalkyl group, a C₂₋₄ aminoalkyl group, a phenyl group optionally substituted by a nitro group, a trifluoromethyl group, an amino group or a C₁₋₄ alkylamino group, a benzyl group optionally substituted by a halogen atom, a C₁₋₄ alkyl group, a C₁₋₄ alkoxy group, a methylenedioxy group or an amino group, a pyridyl group, a thienyl group, a furyl group or a group

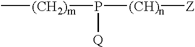

in which m and n independently of one another are integers of 1 to 3, P is oxygen or a group —NH—, Q is hydrogen or a methyl group and Z is a methyl group, a group —OR or —NRR′, where R and R′ independently of one another may be hydrogen, a methyl group or an ethyl group; where R¹⁴ is hydrogen, R¹⁵ may also represent C₁₋₄-alkylamino, and R¹⁸ is a linear or branched C₁₋₆ alkyl group, a C₁₋₄ hydroxyalkyl group, a C₁₋₄ aminoalkyl group, an N—C₁₋₄-alkyl-C₁₋₄-aminoalkyl group, an N,N—C₁₋₄-dialkyl-C₁₋₄-aminoalkyl group, an N—C₁₋₄-hydroxyalkyl-C₁₋₄-aminoalkyl group, a C₁₋₄-alkoxymethyl group, a phenyl group optionally substituted by a halogen atom, a C₁₋₄ alkyl group, a C₁₋₄ alkoxy group, a nitro group, a trifluoromethyl group, an amino group or a C₁₋₄ alkylamino group, a benzyl group optionally substituted by a halogen atom, a C₁₋₄ alkyl group, a C₁₋₄ alkoxy group, an amino group, a trifluoromethyl group, a C₁₋₄-alkylamino group or a nitro group, a heterocycle selected from thiophene, furane and pyridine or a group —(CH₂)ₚ—O—(CH₂)_{q}—OR″, where R″ is hydrogen or a methyl group and p and q are integers which independently of one another are in the range from 1 to 3, or hydrogen with the proviso that, in this case, R¹³ is not an unsubstituted or substituted benzyl group.

The primary intermediates according to the invention are preferably selected from 4,5-diaminopyrazole, 4,5-diamino-1-(2′-hydroxyethyl)-pyrazole, 1-benzyl-4,5-diamino-3-methylpyrazole, 4,5-diamino-1-(2′-hydroxyethyl)-3-(4′-methoxyphenyl)-pyrazole, 4,5-diamino-1-(2′-hydroxyethyl)-3-(4′-methylphenyl)-pyrazole, 4, 5-diamino-1-(2′-hydroxyethyl)-3-(3′-methylphenyl)-pyrazole, 4,5-diamino-3-methyl-1-isopropyl-pyrazole, 4,5-diamino-3-(4′-methoxyphenyl)-1-isopropylpyrazole, 4,5-diamino-1-ethyl-3-methylpyrazole, 4,5-d iamino-1-ethyl-3-(4′-methoxyphenyl)-pyrazole, 4,5-diamino-3-hydroxymethyl-1-methylpyrazole, 4,5-diamino-1-ethyl-3-hydroxymethylpyrazole, 4,5-diamino-3-hydroxymethyl-1-isopropylpyrazole, 4,5-diamino-3-hydroxymethyl-1-tert.butylpyrazole, 4,5-diamino-3-hydroxymethyl-1-phenylpyrazole, 4,5-diamino-3-hydroxymethyl-1-(2′-methoxyphenyl)-pyrazole, 4,5-diamino-3-hydroxymethyl-1-(3′-methoxyphenyl)-pyrazole, 4,5-diamino-3-hydroxymethyl-1-(4′-methoxyphenyl)-pyrazole, 1-benzyl-4,5-diamino-3-hydroxymethylpyrazole, 4,5-diamino-3-methyl-1-(2′-methoxyphenyl)-pyrazole, 4,5-diamino-3-methyl-1-(3′-methoxyphenyl)-pyrazole, 4,5-diamino-3-methyl-1-(4′-methoxyphenyl)-pyrazole, 3-aminomethyl-4,5-diamino-1-methylpyrazole, 3-aminomethyl-4,5-diamino-1-ethylpyrazole, 3-aminomethyl-4,5-diamino-1-isopropylpyrazole, 3-aminomethyl-4,5-diamino-1-tert.butylpyrazole, 4,5-diamino-3-dimethylaminomethyl-1-methylpyrazole, 4,5-diamino-3-dimethylaminomethyl-1-ethylpyrazole, 4,5-diamino-3-dimethylaminomethyl-1-isopropylpyrazole, 4,5-diamino-3-dimethylaminomethyl-1-tert.butylpyrazole, 4,5-diamino-3-ethylaminomethyl-1-methylpyrazole, 4,5-diamino-3-ethylaminomethyl-1-ethylpyrazole, 4,5-diamino-3-ethylaminomethyl-1-isopropylpyrazole, 4,5-diamino-3-ethylaminomethyl-1-tert.butylpyrazole, 4,5-diamino-3-methylaminomethyl-1-methylpyrazole, 4,5-diamino-3-methylaminomethyl-1-isopropylpyrazole, 4,5-diamino-1-ethyl-3-methylaminomethylpyrazole, 1-tert.butyl-4,5-diamino-3-methylaminomethylpyrazole, 4,5-diamino-3-[(2′-hydroxyethyl)aminomethyl]-1-methylpyrazole, 4,5-diamino-3-[(2′-hydroxyethyl)aminomethyl]-1-isopropylpyrazole, 4,5-diamino-1-ethyl-3-[(2′-hydroxyethyl)aminomethyl]-pyrazole, 1-tert.butyl-4,5-diamino-3-[(2′-hydroxyethyl)aminomethyl]-pyrazole, 4-amino-5-(2′-hydroxyethyl)amino-1,3-dimethylpyrazole, 4-amino-5-(2′-hydroxyethyl)-amino-1-isopropyl-3-methylpyrazole, 4-amino-5-(2′-hydroxyethyl)amino-1-ethyl-3-methylpyrazole, 4-amino-5-(2′-hydroxyethyl)amino-1-tert.butyl-3-methyl pyrazole, 4-amino-5-(2′-hydroxyethyl)amino-1-phenyl-3-methylpyrazole, 4-amino-5-(2′-hydroxyethyl)amino-(2-methoxyphenyl)-3-methylpyrazole, 4-amino-5-(2′-hydroxyethyl)amino-1-(3-methoxyphenyl)-3-methylpyrazole, 4-amino-5-(2′-hydroxyethyl)amino-1-(4-methoxyphenyl)-3-methylpyrazole, 4-amino-5-(2′-hydroxyethyl)amino-1-benzyl-3-methylpyrazole, 4-amino-1-ethyl-3-methyl-5-methylaminopyrazole, 4-amino-1-tert-butyl-3-methyl-5-methylaminopyrazole, 4,5-diamino-1,3-dimethylpyrazole, 4,5-diamino-3-tert. butyl-1-methylpyrazole, 4,5-diamino-1-tert. butyl-3-methylpyrazole, 4,5-diamino-1-methyl-3-phenyl-pyrazole, 4,5-diamino-1-(2′-hydroxyethyl)-3-methylpyrazole, 4,5-diamino-1-(2′-hydroxyethyl)-3-phenylpyrazole, 4,5-diamino-1-methyl-3-(2′-chlorophenyl)-pyrazole, 4,5-diamino-1-methyl-3-(4′-chlorophenyl)-pyrazole, 4,5-diamino-1-methyl-3-(3′-trifluoromethylphenyl)-pyrazole, 4,5-diamino-1,3-diphenylpyrazole, 4,5-diamino-3-methyl-1-phenylpyrazole, 4-amino-1,3-dimethyl-5-phenylaminopyrazole, 4-amino-1-ethyl-3-methyl-5-phenylaminopyrazole, 4-amino-1,3-dimethyl-5-methylaminopyrazole, 4-amino-3-methyl-1-isopropyl-5-methylaminopyrazole, 4-amino-3-isobutoxymethyl-1-methyl-5-methylaminopyrazole, 4-amino-3-methoxyethoxymethyl-1-methyl-5-methylaminopyrazole, 4-amino-3-hydroxymethyl-1-methyl-5-methylaminopyrazole, 4-amino-1,3-diphenyl-5-phenylaminopyrazole, 4-amino-3-methyl-5-methylamino-1-phenylpyrazole, 4-amino-1,3-dimethyl-5-hydrazinopyrazole, 5-amino-3-methyl-4-methylamino-1-phenylpyrazole, 5-amino-1-methyl-4-(N,N-methylphenyl)-amino-3-(4′-chlorophenyl)-pyrazole, 5-amino-3-ethyl-1-methyl-4-(N, N-methylphenyl)-aminopyrazole, 5-amino-1-methyl-4-(N ,N-methylphenyl)-amino-3-phenylpyrazole, 5-amino-3-ethyl-4-(N,N-methylphenyl)-aminopyrazole, 5-amino-4-(N,N-methylphenyl)amino-3-phenylpyrazole, 5-amino-4-(N,N-methylphenyl)-amino-3-(4′-methylphenyl)-pyrazole, 5-amino-3-(4′-chlorophenyl)-4-(N,N-methylphenyl)aminopyrazole, 5-amino-3-(4′-methoxyphenyl)-4-(N,N-methylphenyl)-aminopyrazole, 4-amino-5-methylamino-3-phenylpyrazole, 4-amino-5-ethylamino-3-phenylpyrazole, 4-amino-5-ethylamino-3-(4′-methylphenyl)-pyrazole, 4-amino-3-phenyl-5-propylaminopyrazole, 4-amino-5-butylamino-3-phenylpyrazole, 4-amino-3-phenyl-5-phenylaminopyrazole, 4-amino-5-benzylamino-3-phenylpyrazole, 4-amino-5-(4′-chlorophenyl)-amino-3-phenylpyrazole, 4-amino-3-(4′-chlorophenyl)-5-phenylaminopyrazole, 4-amino-3-(4′-methoxyphenyl)-5-phenylaminopyrazole, 1-(4′-chlorobenzyl)-4,5-diamino-3-methylpyrazole, 4,5-diamino-3-hydroxymethyl-1-isopropylpyrazole, 4-amino-1-ethyl-3-methyl-5-methylaminopyrazole, 4-amino-5-(2′-aminoethyl)-amino-1,3-dimethylpyrazole, 4,5-diamino-1-(4′-methoxybenzyl)-pyrazole, 4,5-diamino-1-(4′-methylbenzyl)-pyrazole, 4,5-diamino-1-(4′-chlorobenzyl)-pyrazole, 4,5-diamino-1-(3′-methoxybenzyl)-pyrazole, 4-amino-1-methyl-5-methylaminopyrazole, 4-amino-5-(2′-hydroxyethyl)-amino-1-methylpyrazole, 4,5-diamino-1-ethylpyrazole, 4,5-diamino-1-isopropylpyrazole, 4-amino-1-(2′-hydroxyethyl)-5-(2′-hydroxyethyl)-aminopyrazole, 4-amino-1-(2′-hydroxyethyl)-5-methylaminopyrazole, 4-amino-5-methylaminopyrazole, 4,5-diamino-1-methylpyrazole, 4,5-diamino-1-benzylpyrazole und 4-amino-1-methyl-5-N,N-dimethylaminopyrazole and mixtures of the above-mentioned primary intermediates.

According to the invention, particularly preferred primary intermediates are selected from 4,5-diaminopyrazole, 4,5-diamino-1-(2′-hydroxyethyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert. butyl-3-methylpyrazole, 4,5-diamino-1-(2′-hydroxyethyl)-3-methylpyrazole, 4,5-diamino-1-ethyl-3-methylpyrazole, 4,5-diamino-1-ethyl-3-(4′-methoxyphenyl)-pyrazole, 4,5-diamino-1-ethyl-3-hydroxymethylpyrazole, 4,5-diamino-3-hydroxmethyl-1-methylpyrazole, 4,5-diamino-3-hydroxymethyl-1-isopropylpyrazole, 4,5-diamino-3-methyl-1-isopropylpyrazole and 4-amino-5-(2′-aminoethyl)-amino-1,3-dimethylpyrazole and mixtures of these primary intermediates.

A primary intermediate eminently suitable for the purposes of the invention as a whole is 4,5-diamino-1-(2′-hydroxyethyl)-pyrazole.

Since all the compounds according to the invention are amino compounds, the known acid addition salts may be prepared from them in the usual way. Accordingly, all statements in this specification and hence the claimed scope of protection relate both to the amino compounds of formulae (I) and (II) present in free form and to their water-soluble physiologically compatible salts. Examples of such salts are the hydrochlorides, the hydrobromides, the sulfates, the phosphates, the acetates, the propionates, the citrates and the lactates, the hydrochlorides being preferred.

In a preferred embodiment, the combinations according to the invention of primary intermediates and special secondary intermediates give an oxidation colorant which contains each of the two components in a quantity of 0.005 to 20% by weight and more particularly 0.1 to 5% by weight, based on the colorant as a whole.

The present invention also relates to a process for coloring keratin fibers, more particularly human hair, in which one of the oxidation colorants according to the invention is applied to the fibers to be colored in a suitable cosmetic preparation and the color is developed with an oxidizing agent either by adding the oxidizing agent to the colorant immediately before application and then applying the two to the fibers or by applying the oxidizing agent to the fibers to be colored at the same time as or immediately after the colorant, leaving the two on the fibers for about 10 to 45 minutes and preferably for 30 minutes and then rinsing them out or washing them out with a shampoo.

Keratin fibers in the context of the present invention include pelts, wool, feathers and, more particularly, human hair. Although the combinations of primary and secondary intermediates according to the invention are particularly suitable as oxidation colorants for coloring keratin fibers, there is basically nothing to prevent them from being used in other fields, particularly in color photography.

The oxidation colorants according to the invention contain the primary and secondary intermediates according to the invention and, if desired, may contain other primary and secondary intermediates.

The primary intermediates used are normally primary aromatic amines containing another free or substituted hydroxy or amino group in the para or ortho position, diaminopyridine derivatives, heterocyclic hydrazones and 2,4,5,6-tetraaminopyrimidine and derivatives thereof.

According to the invention, preferred primary intermediates are p-phenylenediamine, p-toluylenediamine, p-aminophenol, o-aminophenol, 1-(2′-hydroxyethyl)-2,5-diaminobenzene, N,N-bis-(2-hydroxyethyl)-p-phenylenediamine, 2-(2,5-diaminophenoxy)-ethanol, 4-amino-3-methylphenol, 2,4,5,6-tetraaminopyrimidine, 2-hydroxy-4,5,6-triaminopyrimidine, 4-hydroxy-2,5,6-triaminopyrimidine, 2,4-dihydroxy-5,6-diaminopyrimidine, 2-dimethylamino-4,5,6-triaminopyrimidine, 2-hydroxymethylamino-4-aminophenol, bis-(4-aminophenyl)-amine, 4-amino-3-fluorophenol, 2-aminomethyl-4-aminophenol, 2-hydroxymethyl-4-aminophenol, 4-amino-2-((diethylamino)-methyl)-phenol, bis-(2-hydroxy-5-aminophenyl)-methane, 1,4-bis-(4-aminophenyl)-diazacycloheptane, 1,3-bis-(N-(2′-hydroxyethyl)-N-(4-aminophenylamino))-2-propanol, 4-amino-2-(2′-hydroxyethoxy)-phenol and 1,10-bis-(2,5-diaminophenyl)-1,4,7,10-tetraoxadecane.

Other particularly preferred primary intermediates are p-phenylenediamine, p-toluylenediamine, p-aminophenol, 1-(2′-hydroxyethyl)-2,5-diaminobenzene, N,N-bis-(2′-hydroxyethyl)-p-phenylenediamine, 4-amino-3-methylphenol, 4-amino-2-((diethylamino)methyl)-phenol, 2-aminomethyl-4-aminophenol, 2,4,5,6-tetraaminopyrimidine, 2-hydroxy-4,5,6-triaminopyrimidine and 4-hydroxy-2,5,6-triaminopyrimidine.

According to the invention, preferred secondary intermediates are

m-aminophenol and derivatives thereof such as, for example, 5-amino-2-methylphenol, 2-hydroxy-4-aminophenoxyethanol, 2,6-dimethyl-3-aminophenol, 5-amino-4-methoxy-2-methylphenol, 5-(2′-hydroxyethyl)-amino-2-methylphenol, 3-(diethylamino)-phenol, N-cyclopentyl-3-aminophenol, 1,3-dihydroxy-5-(methylamino)-benzene and 3-(ethylamino)-4-methylphenol,

o-aminophenol and derivatives thereof,

m-diaminobenzene and derivatives thereof such as, for example, 2,4-diaminophenoxyethanol, 1,3-bis-(2,4-diaminophenoxy)-propane, 1-methoxy-2-amino-4-(2′-hydroxyethylamino)-benzene, 1,3-bis-(2,4-diaminophenyl)-propane, 2,6-bis-(2′-hydroxyethylamino)-1-methylbenzene and 1-amino-3-bis-(2′-hydroxyethyl)-aminobenzene,

o-diaminobenzene and derivatives thereof such as, for example, 3,4-diaminobenzoic acid and 2,3-diamino-1-methylbenzene,

di- and trihydroxybenzene derivatives such as, for example, resorcinol, resorcinol monomethyl ether, 2-methyl resorcinol, 5-methyl resorcinol, 2,5-dimethyl resorcinol, 2-chlororesorcinol, 4-chlororesorcinol, pyrogallol and 1,2,4-trihydroxybenzene,

pyridine derivatives such as, for example, 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine and 3,5-diamino-2,6-dimethoxypyridine,

naphthalene derivatives such as, for example, 1-naphthol, 2-methyl-1-naphthol, 2-hydroxymethyl-1-naphthol, 2-hydroxyethyl-1-naphthol, 1,5-dihydroxynaphthalene, 1,6-dihdroxynaphthalene, 1,7-dihdroxynaphthalene, 1,8-dihdroxynaphthalene, 2,7-dihdroxynaphthalene and 2,3-dihdroxynaphthalene,

morpholine derivatives such as, for example, 6-hydroxybenzomorpholine and 6-aminobenzomorpholine,

quinoxaline derivatives such as, for example, 6-methyl-1,2,3,4-tetrahydroquinoxaline,

pyrazole derivatives such as, for example, 1-phenyl-3-methylpyrazol-5-one,

indole derivatives such as, for example, 4-hydroxyindole, 6-hydroxyindole and 7-hydroxyindole,

methylenedioxybenzene derivatives such as, for example, 1-hydroxy-3,4-methylenedioxybenzene, 1-amino-3,4-methylenedioxybenzene and 1-(2′-hydroxyethyl)-amino-3,4-methylenedioxybenzene.

Particularly preferred secondary intermediates are 1-naphthol, 1,5-, 2,7- and 1,7-dihydroxynaphthalene, 3-aminophenol, 5-amino-2-methylphenol, 2-amno-3-hydroxypyridine, resorcinol, 4-chlororesorcinol, 2-methylresorcinol, 5-methylresorcinol, 2,5-dimethylresorcinol and 2,6-dihydroxy-3,4-dimethylpyridine.

To obtain special shades, oxidation colorants are also often used in combination with substative dyes.

Substantive dyes are typically nitrophenylenediamines, nitroaminophenols, azo dyes, anthraquinones or indophenols. Preferred substantive dyes are the compounds known under the International names or commercial names of HC Yellow 2, HC Yellow 4, HC Yellow 5, HC Yellow 6, Basic Yellow 57, HC Orange 1, Disperse Orange 3, HC Red 1, HC Red 3, HC Red 13, HC Red BN, Basic Red 76, HC Blue 2, HC Blue 12, Disperse Blue 3, Basic Blue 7, Basic Blue 99, HC Violet 1, Disperse Violet 1, Disperse Violet 4, Basic Violet 14, Acid Violet 43, Disperse Black 9, Acid Black 52, Basic Brown 16 and Basic Brown 17 and also 1,4-bis-(2′-hydroxyethyl)-amino-2-nitrobenzene, 3-nitro-4-(2′-hydroxyethyl)-aminophenol, 4-amino-2-nitrodiphenylamine-2′-carboxylic acid, 6-nitro-1,2,3,4-tetrahydroquinoxaline, 2-hydroxy-1,4-naphthoquinone, hydroxyethyl-2-nitrotoluidine, picramic acid, 2-amino-6-chloro-4-nitrophenol, 4-ethylamino-3-nitrobenzoic acid and 2-chloro-6-ethylamino-1-hydroxy-4-nitrobenzene. The colorants according to the invention in this embodiment preferably contain the substantive dyes in a quantity of 0.01 to 20% by weight, based on the colorant as a whole.

The preparations according to the invention may also contain naturally occurring dyes such as, for example, henna red, henna neutral, henna black, camomile blossom, sandalwood, black tea, black alder bark, sage, logwood, madder root, catechu, sedre and alkanet.

Other dye components present in the colorants according to the invention include indoles and indolines and physiologically compatible salts thereof. Preferred examples are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole, 6-aminoindole and 4-aminoindole. Other preferred examples are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid, 6-hydroxyindoline, 6-aminoindoline and 4-aminoindoline.

The oxidation dye precursors or the substantive dyes do not have to be single compounds. On the contrary, other components may be present in small quantities in the hair colorants according to the invention due to the processes used to produce the individual dyes providing these other components do not adversely affect the coloring result or have to be ruled out for other reasons, for example toxicological reasons.

So far as the dyes suitable for use in the hair colorants and tinting compositions according to the invention are concerned, reference is also expressly made to the work by Ch. Zviak, The Science of Hair Care, Chapter 7 (pages 248-250; substantive dyes) and Chapter 8, pages 264-267; oxidation dye precursors), published as Volume 7 of the Series “Dermatology” (Ed.: Ch. Culnan and H. Maibach), Marcel Dekker Inc., New York/Basel, 1986, and to the “Europäische Inventar der Kosmetik-Rohstoffe” published by the Europäische Gemeinschaft and available on floppy disk from the Bundesverband Deutscher Industrie- und Handelsunternehmen für Arzneimittel, Reformwaren und Körperpflegemittel d.V., Mannheim.

Hair colorants are normally adjusted to a mildly acidic to alkaline pH, i.e. to a pH of about 5 to 11, particularly where coloring is carried out oxidatively with atmospheric oxygen or other oxidizing agents, such as hydrogen peroxide. To this end, the colorants contain alkalizing agents, normally alkali metal or alkaline earth metal hydroxides, ammonia or organic amines. Preferred alkalizing agents are monoethanolamine, monoisopropanolamine, 2-amino-2-methylpropanol, 2-amino-2-methylpropane-1,3-diol, 2-amino-2-ethylpropane-1,3-diol, 2-amino-2-methylbutanol and triethanolamine and alkali metal and alkaline earth metal hydroxides. Within this group, monoethanolamine, triethanolamine and 2-amino-2-methylpropanol and 2-amino-2-methylpropane-1,3-diol are preferred. ω-Amino acids, such as ω-aminocaproic acid, may also be used as alkalizing agents.

To form the actual hair colors from the oxidation dye precursors, typical oxidizing agents such as, in particular, hydrogen peroxide or adducts thereof with urea, melamine or sodium borate may be used. However, oxidation with atmospheric oxygen as sole oxidizing agent may be preferred. Oxidation may also be carried out with enzymes. In this case, the enzymes may be used both to produce oxidizing per compounds and to enhance the effect of an oxidizing agent present in small quantities. Thus, the enzymes (enzyme class 1: oxidoreductases) are capable of transferring electrons from suitable primary intermediates (reducing agents) to atmospheric oxygen. Preferred enzymes are oxidases, such as tyrosinase and laccase, although glucoseoxidase, uricase or pyruvate oxidase may also be used. Mention is also made of the procedure whereby the effect of small quantities (for example 1% and less, based on the composition as a whole) of hydrogen peroxide is strengthened by peroxidases.

The preparation of the oxidizing agent is preferably mixed with the preparation of the oxidation dye precursors immediately before coloring of the hair. The ready-to-use hair coloring preparation formed should preferably have a pH value in the range from 6 to 10. In a particularly preferred embodiment, the hair colorant is used in a mildly alkaline medium. The application temperatures may be in the range from 15 to 40° C. but are preferably at the temperature of the scalp. After a contact time of about 5 to 45 and preferably 15 to 30 minutes, the hair colorant is removed from the hair to be colored by rinsing. There is no need for the hair to be washed with a shampoo where a carrier of high surfactant content, for example a coloring shampoo, has been used.

In the particular case of hair which is difficult to color, the preparation containing the oxidation dye precursors may be applied to the hair without preliminary mixing with the oxidation component. The oxidation component is applied after a contact time of 20 to 30 minutes, optionally after rinsing. After another contact time of 10 to 20 minutes, the hair is rinsed and, if desired, shampooed. In a first variant of this embodiment where the preliminary application of the dye precursors is intended to improve penetration into the hair, the corresponding formulation is adjusted to a pH value of about 4 to 7. In a second variant, oxidation with air is initially carried out, the formulation applied preferably having a pH value of 7 to 10. In the subsequent accelerated post-oxidation phase, it can be of advantage to use acidified peroxydisulfate solutions as the oxidizing agent.

Whichever of the processes mentioned above is used to apply the colorant according to the invention, development of the color may be supported and enhanced by adding certain metal ions to the colorant. Examples of such metal ions are Zn²⁺, Cu²⁺, Fe²⁺, Fe³⁺, Mn²⁺, Mn⁴⁺, Li⁺, Mg²⁺, Ca²⁺ and Al³⁺. Zn²⁺, Cu²⁺ and Mn²⁺ are particularly suitable. Basically, the metal ions may be used in the form of a physiologically compatible salt. Preferred salts are the acetates, sulfates, halides, lactates and tartrates. Development of the hair color can be accelerated and the color tone can be influenced as required through the use of these metal salts.

To produce the colorants according to the invention, the oxidation dye precursors are incorporated in a suitable water-containing carrier. For coloring hair, such carriers are, for example, creams, emulsions, gels or even surfactant-containing foaming solutions, for example shampoos, foam aerosols or other formulations suitable for application to the hair.

The colorants according to the invention may also contain any of the known active substances, additives and auxiliaries typical of such formulations. In many cases, the colorants contain at least one surfactant, both anionic and zwitterionic, ampholytic, nonionic and cationic surfactants being suitable in principle. In many cases, however, it has been found to be of advantage to select the surfactants from anionic, zwitterionic or nonionic surfactants.

Suitable anionic surfactants for the preparations according to the invention are any anionic surface-active substances suitable for use on the human body. Such substances are characterized by a water-solubilizing anionic group such as, for example, a carboxylate, sulfate, sulfonate or phosphate group and a lipophilic alkyl group containing around 10 to 22 carbon atoms. In addition, glycol or polyglycol ether groups, ester, ether and amide and hydroxyl groups may also be present in the molecule. The following are examples of suitable anionic surfactants—in the form of the sodium, potassium and ammonium salts and the mono-, di- and trialkanolammonium salts containing 2 or 3 carbon atoms in the alkanol group:

linear fatty acids containing 10 to 22 carbon atoms (soaps),

ether carboxylic acids corresponding to the formula R-O—(CH₂—CH₂O)ₓ—CH₂—COOH, in which R is a linear alkyl group containing 10 to 22 carbon atoms and x=0 or 1 to 16,

amidoether carboxylates corresponding to the formula: [R—NH(—CH₂—CH₂—O)ₙ—CH₂—COO]ₘZ, in which R is a linear or branched, saturated or unsaturated acyl group containing 2 to 29 carbon atoms, n is an integer of 1 to 10, m is the number 1 or 2 and Z is a cation from the group of alkali or alkaline earth metals,

acyl sarcosides containing 10 to 18 carbon atoms in the acyl group,

acyl taurides containing 10 to 18 carbon atoms in the acyl group,

acyl isethionates containing 10 to 18 carbon atoms in the acyl group,

sulfosuccinic acid mono- and dialkyl esters containing 8 to 18 carbon atoms in the alkyl group and sulfosuccinic acid monoalkyl polyoxyethyl esters containing 8 to 18 carbon atoms in the alkyl group and 1 to 6 oxyethyl groups,

linear alkane sulfonates containing 12 to 18 carbon atoms,

linear α-olefin sulfonates containing 12 to 18 carbon atoms,

α-sulfofatty acid methyl esters of fatty acids containing 12 to 18 carbon atoms,

alkyl sulfates and alkyl polyglycol ether sulfates corresponding to the formula R—O(CH₂—CH₂O)ₓ—SO₃H, in which R is a preferably linear alkyl group containing 10 to 18 carbon atoms and x=0 or 1 to 12,

mixtures of surface-active hydroxysulfonates according to DE-A-37 25 030,

sulfated hydroxyalkyl polyethylene and/or hydroxyalkylene propylene glycol ethers according to DE-A-37 23 354,

sulfonates of unsaturated fatty acids containing 12 to 24 carbon atoms and 1 to 6 double bonds according to DE-A-39 26 344,

esters of tartaric acid and citric acid with alcohols in the form of addition products of around 2 to 15 molecules of ethylene oxide and/or propylene oxide with fatty alcohols containing 8 to 22 carbon atoms,

coconut monoglyceride sulfates.

Preferred anionic surfactants are alkyl sulfates, alkyl polyglycol ether sulfates and ether carboxylic acids containing 10 to 18 carbon atoms in the alkyl group and up to 12 glycol ether groups in the molecule and sulfosuccinic acid mono- and dialkyl esters containing 8 to 18 carbon atoms in the alkyl group and sulfosuccinic acid monoalkyl polyoxyethyl esters containing 8 to 18 carbon atoms in the alkyl group and 1 to 6 oxyethyl groups and soaps.

Nonionic surfactants contain, for example, a polyol group, a polyalkylene glycol ether group or a combination of polyol and polyglycol ether groups as the hydrophilic group. Examples of such compounds are

products of the addition of 2 to 30 moles of ethylene oxide and/or 0 to 5 moles of propylene oxide onto linear fatty alcohols containing 8 to 22 carbon atoms, onto fatty acids containing 12 to 22 carbon atoms and onto alkylphenols containing 8 to 15 carbon atoms in the alkyl group,

C₁₂₋₂₂ fatty acid monoesters and diesters of products of the addition of 1 to 30 moles of ethylene oxide onto glycerol,

C₈₋₂₂ alkyl mono- and oligoglycosides and ethoxylated analogs thereof,

products of the addition of 5 to 60 moles of ethylene oxide onto castor oil and hydrogenated castor oil,

products of the addition of ethylene oxide onto sorbitan fatty acid esters and

products of the addition of ethylene oxide onto fatty acid alkanolamides.

Preferred nonionic surfactants are alkyl polyglycosides corresponding to the general formula RO—(Z)ₓ. These compounds are characterized by the following parameters.

The alkyl group R contains 6 to 22 carbon atoms and may be both linear and branched. Primary linear and 2-methyl-branched aliphatic groups are preferred. Such alkyl groups are, for example, 1-octyl, 1-decyl, 1-lauryl, 1-myristyl, 1-cetyl and 1-stearyl. 1-Octyl, 1-decyl, 1-lauryl and 1-myristyl are particularly preferred. Where so-called “oxo alcohols” are used as starting materials, compounds with an odd number of carbon atoms in the alkyl chain predominate.

The alkyl polyglyosides suitable for use in accordance with the invention may, for example, contain only one particular alkyl group R. However, such compounds are normally prepared from natural fats and oils or mineral oils. In this case, mixtures corresponding to the starting compounds or corresponding to the particular working up of these compounds are present as the alkyl groups R.

Particularly preferred alkyl polyglycosides are those in which R consists

essentially of C₈ and C₁₀ alkyl groups,

essentially of C₁₂ and C₁₄ alkyl groups,

essentially of C₈ to C₁₆ alkyl groups or

essentially of C₁₂ to C₁₆ alkyl groups.

Any mono- or oligosaccharides may be used as the sugar unit Z. Sugars containing 5 or 6 carbon atoms and the corresponding oligosaccharides are normally used. Examples of such sugars are glucose, fructose, galactose, arabinose, ribose, xylose, lyxose, allose, altrose, mannose, gulose, idose, talose and sucrose. Preferred sugar units are glucose, fructose, galactose, arabinose and sucrose; glucose is particularly preferred.

The alkyl polyglycosides suitable for use in accordance with the invention contain on average 1.1 to 5 sugar units. Alkyl polyglycosides with x values of 1.1 to 1.6 (also known as alkyl oligoglycosides) are preferred. Alkyl oligoglycosides where x is 1.1 to 1.4 are most particularly preferred.

Besides acting as surfactants, the alkyl polyglycosides or alkyl oligoglycosides may also be used to improve the fixing of perfume components to the hair. Accordingly, in cases where the effect of the perfume oil on the hair is intended to last longer than the duration of the hair treatment, alkyl poly- or oligoglycosides will preferably be used as another ingredient of the preparations according to the invention.

Alkoxylated homologs of the alkyl polyglycosides mentioned may also be used in accordance with the invention. These homologs may contain on average up to 10 ethylene oxide and/or propylene oxide units per alkyl glycoside unit.

Zwitterionic surfactants may also be used, particularly as co-surfactants. In the context of the invention, zwitterionic surfactants are surface-active compounds which contain at least one quaternary ammonium group and at least one —COO⁽⁻⁾ or —SO₃⁽⁻⁾ group in the molecule. Particularly suitable zwitterionic surfactants are the so-called betaines, such as N-alkyl-N,N-dimethyl ammonium glycinates, for example cocoalkyl dimethyl ammonium glycinate, N-acylaminopropyl-N,N-dimethyl ammonium glycinates, cocoacylaminopropyl dimethyl ammonium glycinate and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. A preferred zwitterionic surfactant is the fatty acid amide derivative known by the INCI name of Cocamidopropyl Betaine.

Also suitable, particularly as co-surfactants, are ampholytic surfactants. Ampholytic surfactants are surface-active compounds which, in addition to a C₈₋₁₈ alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids containing around 8 to 18 carbon atoms in the alkyl group. Particularly preferred ampholytic surfactants are N-cocoalkyl aminopropionate, cocoacyl aminoethyl aminopropionate and C₁₂₋₁₈ acyl sarcosine.

Examples of the cationic surfactants suitable for use in the hair treatment preparations according to the invention are, in particular, quaternary ammonium compounds, esterquats and amidoamines.

Preferred quaternary ammonium compounds are ammonium halides, more particularly chlorides and bromides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride and the imidazolium compounds known under the INCI names of Quaternium-27 and Quaternium-83. The long alkyl chains of the above-mentioned surfactants preferably contain 10 to 18 carbon atoms.

Esterquats are known substances which contain both at least one ester function and at least one quaternary ammonium group as structural element. Preferred esterquats are quaternized ester salts of fatty acids with triethanolamine, quaternized ester salts of fatty acids with diethanol alkylamines and quaternized ester salts of fatty acids with 1,2-dihydroxypropyl dialkylamines. Such products are marketed, for example, under the names of Stepantex®, Dehyquart® and Armocare®. The products Armocare® VGH-70, an N,N-bis-(2-palmitoyloxyethyl)-dimethyl ammonium chloride, and Dehyquart® F-75 and Dehyquart® AU-35 are examples of such esterquats.

The alkyl amidoamines are normally prepared by amidation of natural or synthetic fatty acids and fatty acid cuts with dialkyl aminoamines. A compound from this group particularly suitable for the purposes of the invention is the stearamidopropyl dimethylamine obtainable under the name of Tegoamid® S 18.

One example of a quatemary sugar derivative suitable for use as a cationic surfactant is the commercially available product Glucquat®100 (INCI name: Lauryl Methyl Gluceth-10Hydroxypropyl Dimonium Chloride).

The compounds containing alkyl groups used as surfactants may be single compounds. In general, however, these compounds are produced from native vegetable or animal raw materials so that mixtures with different alkyl chain lengths dependent upon the particular raw material are obtained.

The surfactants representing addition products of ethylene and/or propylene oxide with fatty alcohols or derivatives of these addition products may be both products with a “normal” homolog distribution and products with a narrow homolog distribution. Products with a “normal” homolog distribution are mixtures of homologs which are obtained in the reaction of fatty alcohol and alkylene oxide using alkali metals, alkali metal hydroxides or alkali metal alcoholates as catalysts. By contrast, narrow homolog distributions are obtained when, for example, hydrotalcites, alkaline earth metal salts of ether carboxylic acids, alkaline earth metal oxides, hydroxides or alcoholates are used as catalysts. The use of products with a narrow homolog distribution can be of advantage.

In a preferred embodiment, the oxidation colorants used in accordance with the invention contain a care component. This care component is preferably selected from the group consisting of cationic polymers, silicones and protein hydrolyzates and derivatives thereof.

A first group of cationic polymers are the so-called “temporarily cationic” polymers. These polymers normally contain an amino group which is present as a quaternary ammonium group and, hence, cationically at certain pH values.

Among the cationic polymers, however, the permanently cationic polymers are preferred. According to the invention, “permanently cationic polymers” are polymers which contain a cationic group irrespective of the pH of the composition. These are generally polymers which contain a quaternary nitrogen atom, for example in the form of an ammonium group. Preferred cationic polymers are, for example,

the quaternized cellulose derivatives commercially available under the names of Celquat® and Polymer JR®. The compounds Celquat® H 100, Celquat® L 200 and Polymer JR® 400 are preferred quaternized cellulose derivatives,

polysiloxanes containing quaternary groups such as, for example, the commercially available products Q2-7224 (manufacturer: Dow Corning; a stabilized trimethyl silyl amodimethicone), Dow Corning® 929 Emulsion (containing a hydroxylamino-modified silicone which is also known as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethyl siloxanes, Quaternium-80),

cationic guar derivatives such as, in particular, the products marketed under the names of Cosmedia® Guar and Jaguar®,

polymeric dimethyl diallyl ammonium salts and copolymers thereof with esters and amides of acrylic acid and methacrylic acid. The products commercially available under the names of Merquat® 100 (poly(dimethyl diallylammonium chloride)) and Merquat® 550 (dimethyl diallylammonium chloride/acrylamide copolymer) are examples of such cationic polymers,

copolymers of vinyl pyrrolidone with quaternized derivatives of dialkylaminoacrylate and methacrylate such as, for example, vinyl pyrrolidone/dimethylamino methacrylate copolymers quaternized with diethyl sulfate. Such compounds are commercially available under the names of Gafquat® 734 and Gafquat® 755,

The vinyl pyrrolidone/vinyl imidazolinium methochloride copolymers commercially available under the name of Luviquat® FC₃₇₀, FC₅₅₀, FC₉₀₅ and HM 552,

quaternized polyvinyl alcohol;

and the polymers containing quaternary nitrogen atoms in the main polymer chain known under the names of polyquaternium 2, polyquaternium 17, polyquaternium 18 and polyquaternium 27.

Other suitable cationic polymers are the polymers known by the names of polyquaternium 24 (commercial product: Quatrisoft® LM 200 for example), polyquaternium 32, polyquaternium 35 and polyquaternium 37 (commercial products: Salcare® SC₉₂ and Salcare® SC₉₅). Also suitable for use in accordance with the invention are the vinyl pyrrolidone copolymers known by the commercial names of Copolymer 845 (manufacturer: ISP), Gaffix® VC₇₁₃ (manufacturer: ISP), Gafquat® ASCP 1011, Gafquat® HS 110, Luviquat® 8155 and Luviquat® MS 370.

According to the invention, preferred cationic polymers are quaternized cellulose derivatives, polymeric dimethyl diallyl ammonium salts, polyquaternium 27 and copolymers thereof and polymers of the polyquaternium 2 type. Cationic cellulose derivatives, more particularly the commercial product Polymer® JR 400, and polymers of the polyquaternium 2 type, more particularly the commercial product Mirapol® A-15, are most particularly preferred cationic polymers.

The cationic polymers are present in the compositions used in accordance with the invention in quantities of preferably 0.05 to 10% by weight, based on the composition as a whole. Quantities of 0.1 to 5% by weight are particularly preferred.

Amphopolymers may also be used as a care component in combination with or alternatively to the cationic polymers. Amphopolymers are amphoteric polymers, i.e. polymers which contain both free amino groups and free —COOH or —SO₃H groups in the molecule and which are capable of forming inner salts, zwitterionic polymers which contain quaternary ammonium groups and —COO— or —SO₃— groups in the molecule and polymers which contain —COOH— or SO₃H groups and quaternary ammonium groups. One example of an amphopolymer suitable for use in accordance with the invention is the acrylate resin commercially available as Amphomer® which is a copolymer of tert.butylaminoethyl methacrylate, N-(1,1,3,3-tetramethylbutyl)-acrylamide and two or more monomers from the group consisting of acrylic acid, methacrylic acid and simple esters thereof. Other preferred amphopolymers consist of unsaturated carboxylic acids (for example acrylic and methacrylic acid), cationically derivatized unsaturated carboxylic acids (for example acrylamidopropyl trimethyl ammonium chloride) and optionally other ionic or nonionic monomers of the type disclosed, for example, in DE-OS 39 29 973 and the prior art literature cited therein. According to the invention, terpolymers of acrylic acid, methyl acrylate and methacrylamidopropyl trimonium chloride, which are commercially available under the name of Merquat® 2001 N, are particularly preferred amphopolymers.

Other care components suitable for use in accordance with the invention are silicone oils and silicone gums, more particularly dialkyl and alkylaryl siloxanes such as, for example, dimethyl polysiloxane and methylphenyl polysiloxane and alkoxylated and quaternized analogs thereof. Examples of such silicones are the products marketed by Dow Corning under the names of DC190, DC200 and DC1401 and the commercial products DC344 and DC345 of Dow Corning, Q2-7224 (manufacturer: Dow Corning; a stabilized trimethyl silyl amodimethicone), Dow Corning® 929 emulsion (containing a hydroxylamino-modified silicone which is also known as amodimethicone), SM-2059 (manufacturer: General Electric), SLM-55067 (manufacturer: Wacker) and Abil® Quat 3270 and 3272 (manufacturer: Th. Goldschmidt; diquaternary polydimethyl siloxanes, quaternium-80) and the commercial product Fancorsil® LIM-1. A suitable anionic silicone oil is the product Dow Corning® 1784.

Protein hydrolyzates are product mixtures obtained by acid-, base- or enzyme-catalyzed degradation of proteins.

According to the invention, protein hydrolyzates of both vegetable and animal origin may be used.

Animal protein hydrolyzates are, for example, elastin, collagen, keratin, silk and milk protein hydrolyzates which may also be present in the form of salts. Such products are marketed, for example, under the trade names Dehylan® (Henkel), Promois® (Interorgana), Collapuron® (Henkel), Nutrilan® (Grünau), Gelita-Sol® (Deutsche Gelatine Fabriken Stoess & Co.), Lexein® (Inolex) and Kerasol® (Croda).

According to the invention, protein hydrolyzates of vegetable origin, for example soya, almond, pea, alga, potato and wheat protein hydrolyzates, are preferably used. Such products are marketed, for example, under the trade names Gluadin® (Henkel), DiaMin® (Diamalt), Lexein® (Inolex) and Crotein® (Croda).

Although protein hydrolyzates as such are preferably used, amino acid mixtures obtained otherwise may optionally be used instead. It is also possible, but less preferred, to use derivatives of the protein hydrolyzates, for example in the form of their fatty acid condensation products or cationically derivatized. Such products are marketed, for example, under the trade names Lamepon® (Henkel), Lexein® (Inolex), Crolastin® (Croda), Crotein® (Croda), Lamequat® and Croquat®.

The protein hydrolyzates or derivatives are present in the oxidation colorants used in accordance with the invention in quantities of preferably 0.1 to 5% by weight, based on the colorant as a whole. Quantities of 0.1 to 2% by weight are particularly preferred.

Besides the oxidizing agent and the other components mentioned above, the oxidation colorants may in principle contain any other components known to the expert for such cosmetic compositions.

Other active substances, auxiliaries and additives are, for example,

thickeners, such as agar agar, guar gum, alginates, xanthan gum, gum arabic, karaya gum, locust bean gum, linseed gums, dextrans, cellulose derivatives, for example methyl cellulose, hydroxyalkyl cellulose and carboxymethyl cellulose, starch fractions and derivatives, such as amylose, amylopectin and dextrins, clays such as, for example, bentonite or fully synthetic hydrocolloids such as, for example, polyvinyl alcohol,

structurants, such as glucose and maleic acid,

hair-conditioning compounds, such as phospholipids, for example soya lecithin, egg lecithin and kephalins,

perfume oils, dimethyl isosorbide and cyclodextrins,

solvents and solubilizers, such as ethanol, isopropanol, ethylene glycol, propylene glycol, glycerol and diethylene glycol,

fiber-structure-improving components, more particularly mono-, di- and oligosaccharides such as, for example, glucose, galactose, fructose, fruit sugar and lactose,

antidandruff agents, such as Piroctone Olamine, Zinc Omadine and Climbazol,

UV filters, more particularly derivatized benzophenones, cinnamic acid derivatives and triazines,

substances for adjusting the pH value, for example typical acids, more particularly edible acids and bases,

active substances, such as panthenol, pantothenic acid, allantoin, pyrrolidone carboxylic acids and salts thereof, bisabolol, plant extracts and vitamins,

cholesterol,

consistency factors, such as sugar esters, polyol esters or polyol alkyl ethers,

fats and waxes, such as spermaceti, beeswax, montan wax, paraffins,

fatty acid alkanolamides,

complexing agents, such as EDTA, NTA and phosphonic acids,

swelling and penetration agents, such as glycerol, propylene glycol monoethyl ether, carbonates, hydrogen carbonates, guanidines, ureas and primary, secondary and tertiary phosphates,

opacifiers, such as latex,

pearlizers, such as ethylene glycol mono- and distearate and PEG-3-distearate,

stabilizers for hydrogen peroxide and other oxidizing agents,

propellents, such as propane/butane mixtures, N₂O, dimethyl ether, CO₂ and air.

Particulars of other optional components and the quantities in which they are used can be found in the relevant reference books known to the expert, for example K. Schrader, Grundlagen und Rezepturen der Kosmetika, 2nd Edition, Hüthig Buch Verlag, Heidelberg, 1989.

The following Examples are intended to illustrate the invention.

### EXAMPLES

All quantities in the Examples are parts by weight.

1. Preparation of the Coloring Cream

| Mixture A | |
|---|---|
| Hydrenol ® D¹ | 8.50 g |
| Lorol ® techn.¹ | 2.00 g |
| Eumulgin ® B2³ | 0.75 g |
| Texapon ® NSO⁴ | 20.00 g |
| Dehyton ® K⁵ | 12.50 g |
| Water | 30.00 g |

¹C₁₆₋₁₈ fatty alcohol (INCI name: Cetearyl Alcohol) (HENKEL)
²C₁₂₋₁₈ fatty alcohol (INCI name: Coconut Alcohol) (HENKEL)
³cetylstearyl alcohol with ca. 20 EO units (INCI name: Ceteareth-20) (HENKEL)
⁴lauryl ether sulfate, sodium salt (ca. 27.5% active substance: INCI name: Sodium Laureth Sulfate) (HENKEL)
⁵N,N-dimethyl-N-(C₈₋₁₈ cocoamidopropyl)-ammonium acetobetaine (ca. 30% active substance; INCI name: Aqua (Water), Cocamidopropyl Betaine) (HENKEL)

The substances Hydrenol® D, Lorol® and Eumulgin® B2 were melted at 80° C., mixed with the water (heated to 80° C.) containing the Texapon® NSO and Dehyton® K and emulsified with vigorous stirring. The emulsion was then cooled with gentle stirring.

| Mixture B | |
|---|---|
| Sodium sulfite | 1.00 g |
| Ammonium sulfate | 1.00 g |
| Dye precursors | 7.5 mmol of each |
| Ammonia (25% solution) | to pH 10.0 |
| Water | 10.00 g |

The dye precursors were dissolved in the water (heated to 50° C.) while the sodium sulfite, ammonium sulfate and ammonia were added.

The dye precursor solution (mixture B) was added to the emulsion (mixture A) and, after adjustment to pH 10 with ammonia, the whole was made up with water to 100 g, followed by stirring until the temperature had reached room temperature.

1.2 Coloring of the Keratin Fibers

The coloring cream thus obtained was mixed with a 3% H₂O₂ solution in a ratio of 2:1 and the mixture was applied to 5 cm long tresses of standardized, 80% gray but not specially pretreated human hair (Kerling). After a contact time of 30 minutes at 32° C., the hair was rinsed, washed with a standard shampoo and then dried.

The following secondary intermediates were used for coloring:

| | |
|---|---|
| K1 | 2-chloro-6-methyl-3-(2′-hydroxyethylamino)-phenol |
| K2 | 2-chloro-6-methyl-3-(carbethoxyamino)-penol |
| K3 | 4-(N′,N′-diethylcarbamoyl)-methylamino-2-hydroxy-3-chlorotoluene |
| K4 | 3-(2′-aminoethylamino)-2-chloro-6-methylphenol |
| K5 | 2-chloro-6-methyl-3-(trifluoroacetylamino)-phenol |

With 4,5-diamino-1-(2′-hydroxyethyl)-pyrazole as primary intermediate, the following colors were obtained:

| Secondary intermediate | Hair color |
|---|---|
| K1 | Cherry red |
| K2 | Jasper red |
| K3 | Gray-magenta |
| K4 | Cherry red |
| K5 | Cherry red |

The colors were distinguished by high fastness to washing and brilliance.

What is claimed:

1. An oxidation colorant composition for coloring keratin-containing fibers comprising a combination of at least one 4,5-diaminopyrazole derivative or a physiologically compatible salt thereof as a primary intermediate and at least one halogenated m-aminophenol or a physiologically compatible salt thereof as a secondary intermediate of the formula 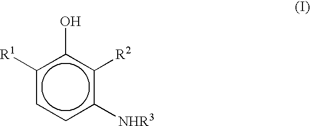
wherein
R¹ is selected from the group consisting of a C₁₋₄ alkyl group, a C₁₋₄ alkoxy group or an allyl group,
R² is a halogen atom and
R³ is selected from the group consisting of
a C₁₋₄ alkyl group,
a C₁₋₄ alkoxy group,
a C₃₋₄ dihydroxyalkyl group,
a C₁₋₄ alkyl group substituted by 1 to 4 halogen atoms,
a methanesulfonyl group,
a group —COOR⁴, where R⁴ is hydrogen or a C₁₋₄ alkyl group,
a group —COR_{f}, where R_{f} is a perfluorinated C₁₋₄ alkyl group,
a group —CONR⁵R⁶ or
a group —(CₙH₂ₙ)—B wherein n=1-4 and B is selected from
a hydroxy group,
a group —NR⁷R⁸ or
a group —CONR⁹R¹⁰, and
R⁵, R⁶, R⁷, R⁸, R⁹ and R¹⁰ independently are selected from the group consisting of hydrogen, a C₁₋₄ alkyl group or a C₁₋₄ monohydroxyalkyl group and R⁵ and R⁶, R⁷ and R⁸ and R⁹ and R¹⁰ together with the nitrogen atom carrying them may also be part of a morpholino, piperidino or pyrrolidino ring.

2. The oxidation colorant composition of claim 1, wherein R³ is selected from the group consisting of
a C₁₋₄ alkyl group,
a trifluoromethyl group,
a methanesulfonyl group,
a group —COOR⁴, where R⁴ is hydrogen or a C₁₋₄ alkyl group,
a group —COR_{f}, where R_{f} is a perfluorinated C₁₋₄ alkyl group,
a group —CONR⁵R⁶ or
a group —(CₙH₂ₙ)—B where n=1-4 and B is selected from
a hydroxy group,
a group —NR⁷R⁸ or
a group —CONR⁹R¹⁰, and
R⁵, R⁶, R⁷, R⁸, R⁹ and R¹⁰ independently of one another are hydrogen or a C₁₋₄ alkyl group.

3. The oxidation colorant composition of claim 2 wherein R¹ is a C₁₋₄ alkyl group or an allyl group.

4. The oxidation colorant composition of claim 3 wherein R² is a chlorine atom.

5. The oxidation colorant composition of claim 4, wherein R¹ is a methyl group.

6. The oxidation colorant composition of claim 5 wherein R³ is selected from the group consisting of —CH₂CH₂OH, —CH₂CH₂NH₂, —CH₂CON(C₂H₅)₂, —COOCH₅ and —COCF₃.

7. The oxidation colorant composition of claim 6, wherein the halogenated m-aminophenol is selected from the group consisting of -chloro-6-methyl-3-(2′-hydroxyethylamino)-phenol, 2-chloro-6-methyl-3-(carbethoxyamino)-phenol, 4-(N′,N′-diethylcarbamoyl)-methylamino-2-hydroxy-3-chlorotoluene, 3-(2′-aminoethylamino)-2-chloro-6-methylphenol and 2-chloro-6-methyl-3-(trifluoroacetylamino)-phenol or a physiologically compatible salt thereof.

8. The oxidation colorant composition of claim 7, wherein the halogenated m-aminophenol is selected from the group consisting of 2-chloro-6-methyl-3-(carbethoxyamino)-phenol, 2-chloro-6-methyl-3-(trifluoroacetylamino)-phenol and 4-(N′,N′-diethylcarbamoyl)-methylamino-2-hydroxy-3-chlorotoluene or a physiologically compatible salt thereof.

9. The oxidation colorant composition of claim 8 wherein the 4,5-diaminopyrazole derivative or a physiologically compatible salt thereof has the formula 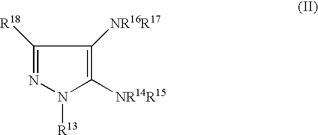
wherein
R¹³, R¹⁴, R¹⁵, R¹⁶ and R¹⁷ independently are selected from the group consisting of hydrogen, a linear or branched C₁₋₆ alkyl group, a C₂₋₄ hydroxyalkyl group, a C₂₋₄ aminoalkyl group, a phenyl group optionally substituted by a nitro group, a trifluoromethyl group, an amino group or a C₁₋₄ alkylamino group, a benzyl group optionally substituted by a halogen atom, a C₁₋₄ alkyl group, a C₁₋₄ alkoxy group, a methylenedioxy group or an amino group, a pyridyl group, a thienyl group, a furyl group or a group of the formula 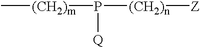
wherein m and n independently are integers of 1 to 3, P is oxygen or the group —NH—, Q is hydrogen or a methyl group and Z is a methyl group, a group —OR or —NRR′, where R and R′ independently of one another may be hydrogen, a methyl group or an ethyl group;
and where R¹⁴ is hydrogen, R¹⁵ may also represent C₁₋₄-alkylamino, and R¹⁸ is selected from the group consisting of a linear or branched C₁₋₆ alkyl group, a C₁₋₄ hydroxyalkyl group, a C₁₋₄ aminoalkyl group, an N—C₁₋₄-alkyl-C₁₋₄-aminoalkyl group, an N,N—C₁₋₄-dialkyl-C₁₋₄-aminoalkyl group, an N—C₁₋₄-hydroxyalkyl-C₁₋₄-aminoalkyl group, a C₁₋₄-alkoxymethyl group, a phenyl group optionally substituted by a halogen atom, a C₁₋₄ alkyl group, a C₁₋₄ alkoxy group, a nitro group, a trifluoromethyl group, an amino group or a C₁₋₄ alkylamino group, a benzyl group optionally substituted by a halogen atom, a C₁₋₄ alkyl group, a C₁₋₄ alkoxy group, an amino group, a trifluoromethyl group, a C₁₋₄-alkylamino group or a nitro group, a heterocycle selected from thiophene, furane and pyridine or a group —(CH₂)ₚ—O—(CH₂)_{q}—OR″, where R″ is hydrogen or a methyl group and p and q are integers independently from 1 to 3, or hydrogen with the proviso that where R¹⁸ is hydrogen then R¹³ is not an unsubstituted or substituted benzyl group.

10. The oxidation colorant composition of claim 9 wherein the 4,5-diaminopyrazole derivative is selected from the group consisting of 4,5-diaminopyrazole, 4,5-diamino-1-(2′-hydroxyethyl)-pyrazole, 4,5-diamino-1,3-dimethylpyrazole, 4,5-diamino-3-methyl-1-phenylpyrazole, 4,5-diamino-1-methyl-3-phenylpyrazole, 1-benzyl-4,5-diamino-3-methylpyrazole, 4,5-diamino-1-tert.butyl-3-methyl-pyrazol, 4,5-diamino-1-(2′-hydroxyethyl)-3-methylpyrazole, 4,5-diamino-1-ethyl-3-methylpyrazole, 4,5-diamino-1-ethyl-3-(4′-methoxyphenyl)-pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole and 4,5-diamino-3-methyl-1-isopropylpyrazole or a physiologically compatible salt thereof.

11. The oxidation colorant composition of claim 10 wherein the 4,5-diaminopyrazole is selected from 4,5-diaminopyrazole or 4,5-diamino-1-(2′-hydroxyethyl)-pyrazole or a physiologically compatible salt thereof.

12. The oxidation colorant composition of claim 11 which further contains at least one other primary intermediate.

13. The oxidation colorant composition of claim 12 the other primary intermediate is selected from the group consisting of p-phenylenediamine, p-toluylenediamine, p-aminophenol, 1-(2′-hydroxyethyl)-2,5-diaminobenzene, N,N-bis-(2′-hydroxyethyl)-p-phenylenediamine, 4-amino-3-methylphenol, 4-amino-2-((diethylamino)-methyl)-phenol, 2-aminomethyl-4-aminophenol, 2,4,5,6-tetraaminopyrimidine, 2-hydroxy-4,5,6-triaminopyrimidine and 4-hydroxy-2,5,6-triaminopyrimidine.

14. The oxidation colorant composition of claim 13 which further contains at least one other secondary intermediate.

15. The oxidation colorant composition of claim 14 wherein the secondary intermediate is selected from the group consisting of 1-naphthol, 1,5-dihydroxynaphthalene, 2,7—dihydroxynaphthalene and 1,7-dihydroxynaphthalene, 3-aminophenol, 5-amino-2-methylphenol, 2-amino-3-hydroxypyridine, resorcinol, 4-chlororesorcinol, 2-methylresorcinol, 5-methylresorcinol, 2,5-dimethylresorcinol and 2,6-dihydroxy-3,4-dimethylpyridine.

16. The oxidation colorant composition of claim 15 wherein the primary intermediates are present in a quantity of 0.005 to 20% by weight and the secondary intermediates are present in a quantity of 0.005 to 20% by weight of the composition.

17. The oxidation colorant composition of claim 16, wherein at least one substantive dye is additionally present.

18. The oxidation colorant composition of claim 16 wherein the primary intermediates are present in a quantity of 0.1 to 5% by weight and the secondary intermediates are present in a quantity of 0.1 to 5% by weight of the composition.

19. A process for coloring keratin fibers comprising applying the oxidation colorant composition of claim 1 in a suitable cosmetic preparation to the fibers to be colored and the color is developed with an oxidizing agent either by adding the oxidizing agent to the colorant composition immediately before application and then applying the two to the fibers or by applying the oxidizing agent to the fibers to be colored at the same time as or immediately after the colorant composition, leaving the two on the fibers for about 10 to 45 minutes and preferably for 30 minutes and then rinsing them out or washing them out with a shampoo.
